(19) United States
(12) Patent Application Publication
(10) Pub. No.: US 2015/0306360 A1
(43) Pub. Date: Oct. 29, 2015

(54) Rotational Localized Drug Delivery Device

(71) Applicant: Boston Scientific Scimed Inc., Maple Grove, MN (US)

(72) Inventors: Brian Joseph Tischler, Shoreview, MN (US); Jaydeep Y. Kokate, Plymouth, MN (US); Derek Kenneth Larson, Golden Valley, MN (US); Christopher J. Clark, St. Michael, MN (US); Gary John Pederson, JR., Albertville, MN (US); Katherine Stryker Brodeen Routh, Coon Rapids, MN (US)

(73) Assignee: Boston Scientific Scimed Inc., Maple Grove, MN (US)

(21) Appl. No.: 14/691,103

(22) Filed: Apr. 20, 2015

Related U.S. Application Data
(60) Provisional application No. 61/983,256, filed on Apr. 23, 2014.

(51) Int. Cl. A61M 25/10; A61M 5/315

(52) U.S. Cl. CPC A61M 25/1002; A61M 5/31586; A61M 25/10184; A61M 2025/105

(57) ABSTRACT

A drug delivery device that uses centrifugal force to deliver a drug to a body lumen wall. The drug delivery device comprises an impeller that includes an impeller shaft; an impeller head at the distal end of the impeller shaft; a drug lumen; an impeller housing; and a housing shaft attached to the impeller housing. In use, the drug is advanced along the drug lumen to the impeller head; at least the impeller head is rotated so that when drug exits the drug lumen, the centrifugal force of the rotating impeller causes the drug to move radially outward from the drug delivery device, towards and into the body lumen wall.

### CROSS-REFERENCE TO RELATED APPLICATIONS

This application claims priority to U.S. Patent Provisional Application No. 61/983,256, filed Apr. 23, 2014, the entire contents of which is hereby incorporated herein by reference.

### STATEMENT REGARDING FEDERALLY SPONSORED RESEARCH

Not Applicable

### BACKGROUND OF THE INVENTION

Drug coated balloons are one mechanism used to deliver a drug to a treatment site. A drug coated balloon deposits a drug onto a vessel wall by the disruption of a coating present on the balloon surface. The effective transport of the drug and its subsequent release at the desired site is enabled by an excipient. An excipient is a non-polymeric additive to a drug-containing layer that facilitates adhesion to the device and/or alters release properties from the device upon placement at a treatment site. However, drug coated balloons have some drawbacks. One drawback is that a majority of the drug and excipient are lost downstream, which potentially has harmful side effects. Another drawback is that since most of the drug is removed from the balloon in the first inflation, drug coated balloons are a single lesion treatment device. This does not allow a physician to treat multiple lesions or a single lesion longer than the balloon length with a single device.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the disclosure is set forth below. Additional details of the summarized embodiments of the present disclosure and/or additional embodiments of the present disclosure may be found in the Detailed Description of the Invention below.

### BRIEF SUMMARY OF THE INVENTION

A drug delivery device 10 of the present disclosure addresses one or more of the drawbacks discussed above with regard to drug coated balloons. In at least one embodiment, the present disclosure is directed to a drug delivery device that delivers a drug to a body lumen wall by a centrifugal force. The drug delivery device comprises an impeller that includes an impeller shaft; an impeller head at the distal end of the impeller shaft; a drug lumen; an impeller housing; and a housing shaft attached to the impeller housing. In use, the drug is advanced along the drug lumen to the impeller head; at least the impeller head is rotated so that when drug exits the drug lumen, the centrifugal force of the rotating impeller causes the drug to move radially outward from the drug delivery device, towards and into the body lumen wall.

In a further aspect, the drug delivery device includes a drug delivery mechanism to advance the drug through the drug lumen to the impeller head.

In a still further aspect, the drug delivery device includes at least one expandable region. The expandable region can be used to block blood flow during delivery of the drug and/or dilates the lumen prior to drug delivery.

In one aspect, the drug delivery device comprises: an impeller, the impeller comprising: an impeller housing positioned at a distal end of a housing shaft, the impeller housing and the housing shaft each defining a lumen, the impeller housing comprising a side opening for passage of a drug therethrough; a rotatable impeller head positioned at a distal end of an impeller shaft, the impeller head positioned in the lumen of the impeller housing, the impeller shaft positioned in the lumen defined by the housing shaft; and a drug lumen for passage of a drug to the rotatable impeller head for delivery into a lumen wall by the rotatable impeller.

In a further aspect of the drug delivery device, the impeller shaft is rotatable.

In a further aspect of the drug delivery device, the side opening is a plurality of side openings.

In a further aspect of the drug delivery device, the impeller head has a textured outer surface.

In a further aspect of the drug delivery device, the textured outer surface is formed by a rib.

In a further aspect of the drug delivery device, the rib is a plurality of ribs.

In a further aspect of the drug delivery device, the impeller further comprises a guidewire lumen for passage of a guidewire.

In a further aspect of the drug delivery device, the guidewire lumen is defined in part by the impeller shaft.

In a further aspect of the drug delivery device, the drug lumen is defined in part by the impeller shaft.

In a further aspect of the drug delivery device, the drug lumen is defined in part by the housing shaft.

In a further aspect of the drug delivery device, the impeller further comprises a drug delivery mechanism to advance the drug to the impeller head.

In a further aspect of the drug delivery device, the drug delivery mechanism is selected from the group consisting of pressurized CO₂ gas; saline; agitated saline; a plunger; a syringe; a slurry; and combinations thereof.

In a further aspect of the drug delivery device, the drug delivery mechanism is a plunger positioned in the drug lumen.

In a further aspect of the drug delivery device, the drug delivery mechanism is a syringe in fluid communication with the drug lumen.

In a further aspect of the drug delivery device, the impeller further comprising a first expandable region positioned proximal to the rotatable impeller head.

In a further aspect of the drug delivery device, the impeller further comprising a second expandable region positioned distal to the rotatable impeller head.

In a further aspect of the drug delivery device, the first and second expandable regions form a part of the impeller housing.

In a further aspect of the drug delivery device, the drug delivery device further comprising an exterior shaft defining a lumen, the housing shaft positioned in the lumen of the exterior shaft, wherein the first expandable region forms a part of the exterior shaft.

In a further aspect of the drug delivery device, the first and second expandable regions are selected from the group consisting of inflatable balloons and electroactive polymers.

In another aspect a method of deliver a drug to a lumen wall comprises: advancing a drug delivery device to a treatment site, the drug delivery device comprising: an impeller, the impeller comprising: an impeller housing positioned at a distal end of a housing shaft, the impeller housing and the housing shaft each defining a lumen, the impeller housing comprising a side opening for passage of a drug therethrough; a rotatable impeller head positioned at a distal end of an impeller shaft, the impeller head positioned in the lumen of the impeller housing, the impeller shaft positioned in the lumen defined by the housing shaft; a drug lumen for passage of a drug to the rotatable impeller head; advancing a drug through the drug lumen to the impeller head; and rotating the impeller head to deliver the drug by centrifugal force.

In a further aspect of the method to deliver a drug to a lumen wall, the drug comprises a crystalline form of the drug.

These and other embodiments are pointed out with particularity in the detailed description, and the claims annexed hereto and forming a part hereof. However, for further understanding reference can be made to the drawings which form a further part hereof and the accompanying descriptive matter, in which one or more embodiments are illustrated and described.

### BRIEF DESCRIPTION OF THE SEVERAL VIEWS OF THE DRAWING(S)

FIG. 1 is an end view of an impeller.

FIG. 2 is a schematic side view of an impeller.

FIG. 3 is a schematic view of a proximal end region of an impeller.

FIG. 4 is a schematic view of an embodiment of the distal end region of a drug delivery device comprising an impeller.

FIG. 5 is a schematic view of an embodiment of the distal end region of a drug delivery device comprising an impeller.

FIG. 6 is a schematic view of an embodiment of the distal end region of a drug delivery device comprising an impeller.

### DETAILED DESCRIPTION OF THE INVENTION

While the subject matter of the present disclosure may be embodied in many different forms, there are described in detail herein specific preferred embodiments of the present disclosure. This description is an exemplification of the principles of the present disclosure and is not intended to limit the present disclosure to the particular embodiments illustrated.

For the purposes of this disclosure, like reference numerals in the figures shall refer to like features unless otherwise indicated.

As used in this application, an “inner surface” is a surface that defines a lumen and the “outer surface” is opposite the inner surface.

As used in this application, “proximal” and “distal” are referenced to the user of the device so that “proximal” is closer to the user than “distal.”

Drug Delivery Device

A drug delivery device 10 of the present disclosure delivers a drug 32 to a body lumen wall by a centrifugal force. The drug delivery device 10 can be used in the cardiovascular system; the gastrointestinal system; the trachea; and elsewhere in the body (e.g., any body lumen). A drug delivery device as disclosed herein can be used at multiple treatment sites or long treatment sites. Additionally, in at least one embodiment, a drug delivery device as disclosed herein minimizes drug lost downstream.

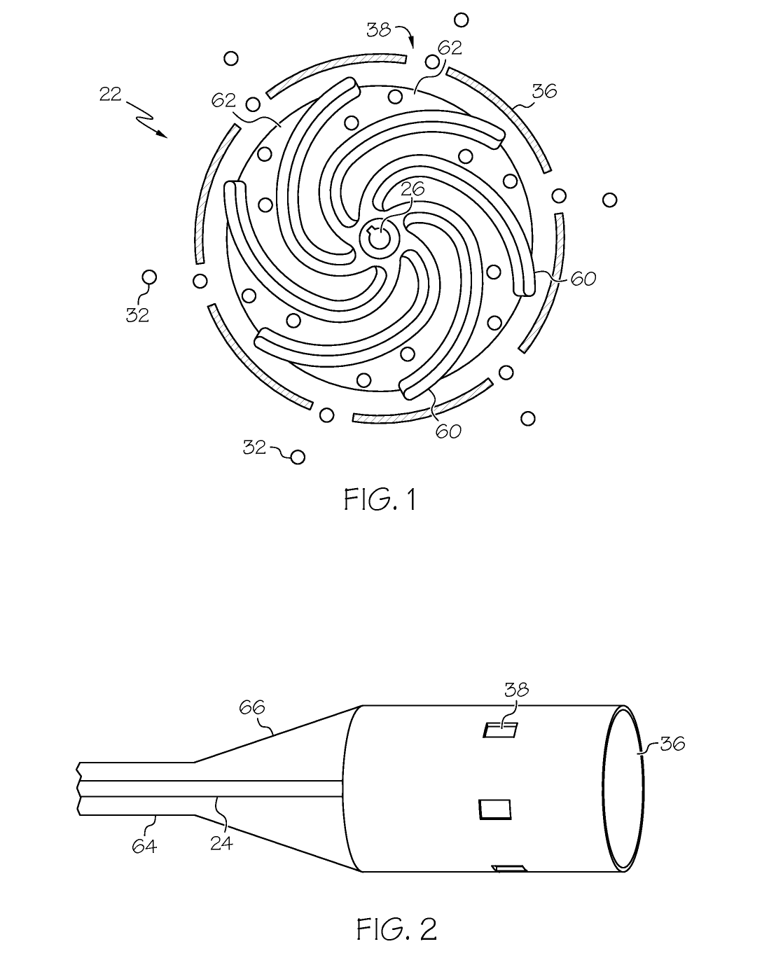

The impeller 20 includes an impeller shaft 24; an impeller head 22 at the distal end of the impeller shaft 24; a drug lumen 26; an impeller housing 36; and a housing shaft 64 attached to the impeller housing 36 (see e.g. FIGS. 1-6). The impeller 20 can be advanced to a target site with or without the use of a guide wire. For impellers advanced to a target site with a guide wire, the impeller 20 over the wire, where the guide wire enters the impeller at the proximal end and extends the longitudinal length of the impeller 20; or the impeller can be monorail, where the guide wire extends through only a portion of the impeller 20; as are known in the art. For impellers advanced to a target site without a guide wire, the impeller can be advanced through a sheath, introducer, or guide catheter, as is known in the art.

In at least one embodiment, the centrifugal force to drive the drug 32 into a target site is provided by an impeller 20 that is configured to rotate. In use, the drug is advanced along the drug lumen to the impeller head 22; at least the impeller head 22 is rotated so that when drug exits the drug lumen 26, the centrifugal force of the rotating impeller 20 causes the drug 32 to move radially outward from the drug delivery device 10, towards and into the body lumen wall 8. In other words, the force imparted from the impeller head to the drug is the primary means by which a majority of the drug enters into the tissue. It is noted that the centrifugal force needed to embed the drug at a desired depth and dosage into the body lumen wall can be empirically determined by research in explanted diseased human superficial femoral arteries (SFAs). For example for balloons coated with drug crystals, 8-12 atm balloon pressure is required to embed drug crystals from the surface of the balloon into the vessel wall. A similar pressure, which is proportional to the centrifugal force, would be targeted for the crystals exiting from the device into the vessel wall. Although it is possible for a minority of the drug to be taken into the tissue by a secondary means after exiting the drug delivery device (e.g. absorption, passive diffusion, facilitated passive diffusion; active transport, and/or pinocytosis), a drug that enters into the tissue by these secondary means, or by any means other than by the force imparted to the drug by the rotating impeller head, is not a drug that is driven, delivered or embedded into the lumen wall or tissue; and is not a drug that penetrates into the lumen wall or tissue, as disclosed for the drug delivery device of the present disclosure.

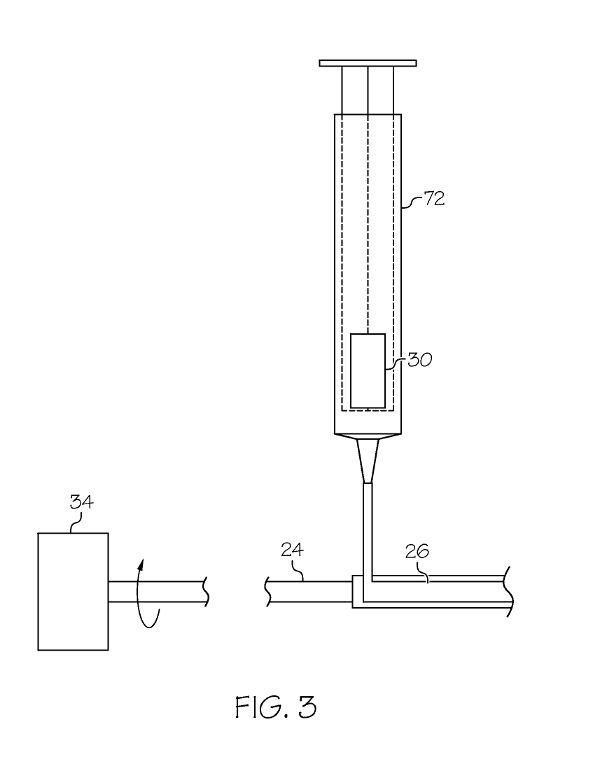

Any suitable mechanism 34 can be used to rotate the impeller 20 at a rate sufficient to provide the desired centrifugal force. In one embodiment, the mechanism 34 is detachably connected to the impeller 20 (e.g. FIG. 3). In another embodiment, the mechanism 34 forms a part of the impeller 20. In other words, the impeller is not detachably connected to the impeller 20. As noted above, the mechanism 34 at least rotates the impeller head 22 of the impeller 20 (as indicated by arrow in FIG. 1). In a further aspect, the mechanism rotates the impeller shaft 24 and the impeller head 22 (as indicated by arrow in FIG. 3). One example of a suitable mechanism 34 is a motor drive with RPM (revolutions per minute) up to 250,000. As used herein, a “motor drive” is a system consisting of an electric or pneumatic motor and accessory parts, used to power machinery. It is noted that the following equation expresses the centrifugal force (Fc) as a function of RPM:

Fc=mrn²

where m=mass (kg); r=radius (m); and n=revolution per minute (rpm).

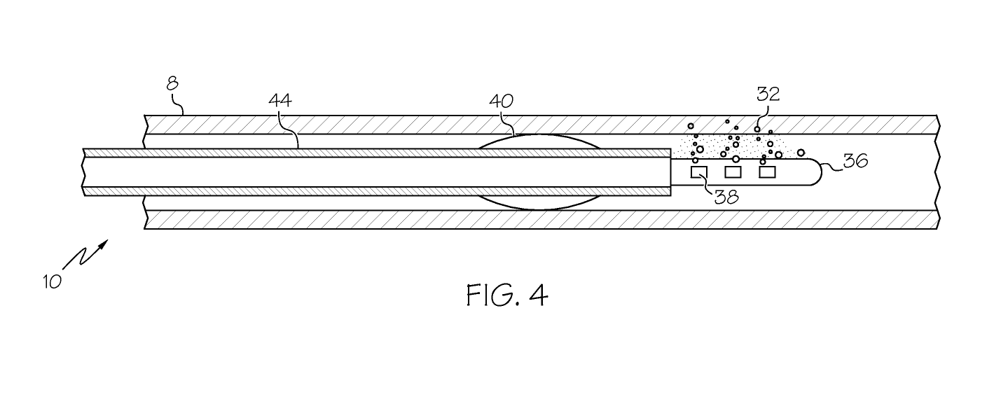

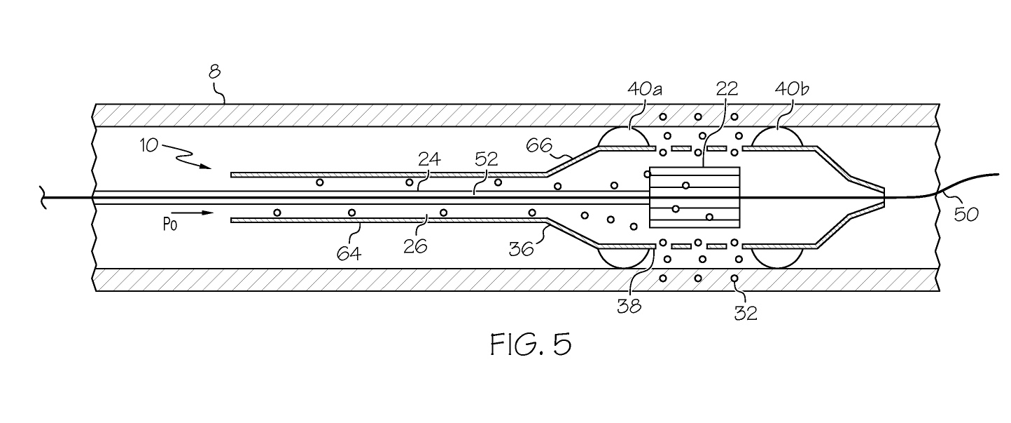

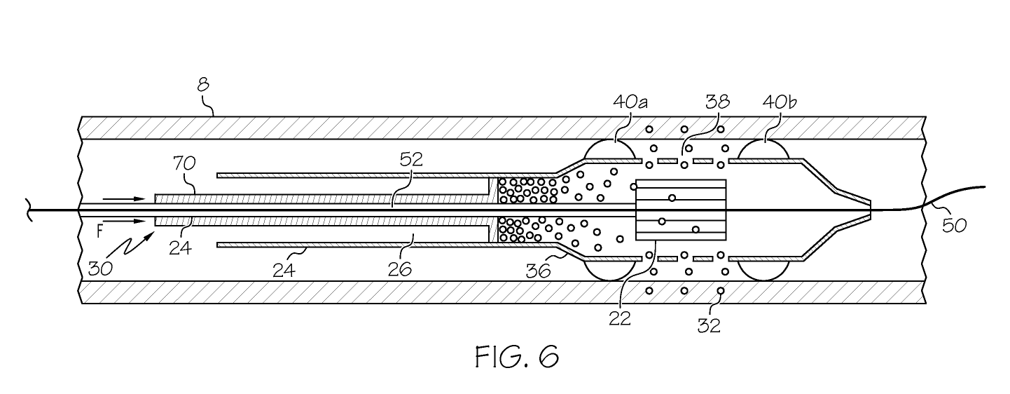

In a further aspect, the impeller 20 defines at least one lumen, such as a guide wire lumen 52 (see e.g. FIGS. 5-6), a drug lumen 26 (see e.g. FIGS. 1 and 4), or a lumen that is a combination guide wire and drug lumen. The lumen can have an open distal end (e.g. the guide wire lumen 52 shown in FIGS. 5-6), or a closed distal end (e.g. the drug lumen of the drug delivery device 10 shown in FIG. 4). In one aspect an impeller 20 with a closed distal end can be advanced to a treatment site through a sheath, guide catheter, introducer, endoscope, or other suitable device without using a guide wire.

The drug lumen 26 can be defined by the impeller shaft 24 (see e.g. FIGS. 1 and 4), the housing shaft 64, or by an exterior shaft 44 that is external to the housing shaft 64 of the impeller 20 (see e.g. FIGS. 5-6). For a drug lumen 26 defined by the impeller shaft 24, the impeller head 22 can define the distal end region and the distal opening of the drug lumen 26 (see e.g. FIG. 1). The shaft 24, 64, 44 defining the drug lumen 26 can be formed of a polymeric material or a metallic material.

The impeller head 22 is positioned at the distal end of the impeller shaft 24. The impeller head 22 has a diameter and a length. The diameter of the impeller head 22 can be the same or different than the diameter of the impeller shaft 24. In some embodiments, the impeller head 22 has a diameter greater than the diameter of the impeller shaft 24 (see e.g. FIG. 5). The length of the impeller head 22 is approximately 1 mm to 10 mm. In one embodiment, the impeller head 22 has a length of approximately 1 mm.

In one further aspect, at least a portion of the outer surface of the impeller head 22 is a textured outer surface for imparting the centrifugal force to the drug 32 in any suitable medium. The impeller head 22 can have any suitable textured outer surface. The textured outer surface can also be provided on the distal face of the impeller head 22. Without being bound by theory, in some embodiments the texture on the outer surface of the impeller head creates friction that encourages rotation of the drug 32 and/or the medium carrying the drug 32. In other embodiments, the texture on the outer surface of the impeller head impacts the drug 32 thereby imparting the centrifugal force to the drug. The longitudinal extent of the textured surface is at most equal to the longitudinal length of the impeller head 22. FIG. 5 shows an example of an impeller head 22 with a textured surface having with a longitudinal extent less than the longitudinal length of the impeller head 22. In one aspect, the textured outer surface of the impeller head 22 includes at least one protuberance 60 (see e.g. FIGS. 1 and 5). In one aspect, the protuberance(s) 60 aid in directing the drug radially outward from the impeller head 22. The protuberance 60 can have any suitable shape and size. Examples of protuberances 60 include but are not limited to ribs, cleats, posts, bumps, and combinations thereof. The protuberances 60 can be provided in any pattern.

FIG. 1 shows an example of a protuberance 60 in the form of a rib. As can be seen in FIG. 1, adjacent ribs 60 define a channel 62 therebetween. In this example, the rib(s) 60 and channel(s) 62 are positioned on the distal face of the impeller head. Each rib 60 has two sides each with a height measured from the outer surface of the impeller head 22; and a top surface that extends between the two sides; and a width. The rib 60 can be straight (see e.g. FIG. 5) or curved (see e.g. FIG. 1). In one embodiment, the curved rib 60 extends helically around the impeller head 22. In at least one embodiment, the ribs curve away from the distal end opening of the drug lumen 26 that is defined in part by the impeller head 22. In this embodiment, the distal face of the impeller head comprises the ribs 60 and the channels 62 (see e.g. FIG. 1).

In another further aspect, the impeller head 22 has a smooth outer surface for imparting the centrifugal force to the drug 32 in a viscous medium. In other words, the outer surface is not textured. In one aspect, the medium has a viscosity of 0.01 to 50,000 cP. Without being bound by theory, rotation of an impeller head with a smooth outer surface creates friction that encourages rotational movement of the medium carrying the drug 32 thereby directing the drug 32 towards the body lumen wall.

The impeller head 22 is positioned inside the lumen of the impeller housing 36. Thus, the impeller head 22 has a diameter less than the interior diameter of the impeller housing 36. The impeller head 22 can be axially/longitudinally fixed relative to the impeller housing 36, or axially/longitudinally moveable relative to the impeller housing 36). In one embodiment the distal end of the impeller housing 36 is distal to the distal end of the impeller head 22. In other words, in this embodiment the impeller head 22 is positioned entirely inside the impeller housing 36. The distal end of the impeller housing 36 can define at least one opening (see e.g. FIGS. 2 and 5-6), or no openings (i.e. have a closed distal end, as shown e.g. in FIG. 4). In one aspect the distal end of the impeller housing 36 defines an opening through which a guidewire extends (see e.g. FIG. 5). In a further aspect, when a guidewire extends through the opening defined by the distal end of the impeller housing, the opening is occluded and no drug can pass through the opening.

The housing shaft 64 extends proximally from the impeller housing 36 to the proximal end region of the impeller 20. The impeller housing serves as a reservoir for the drug as well as providing a structure for the impeller to safely and reliably rotate without damaging adjacent vascular tissue. The impeller housing 36 has a longitudinal length, a diameter, and a wall that defines a lumen and at least one side opening 38 sized for the drug to exit the impeller housing 36 (see e.g. FIG. 1).

The length and diameter of the impeller housing controls the pressure inside of the system and the centrifugal force. The diameter of the impeller housing is designed to navigate within the vasculature and ideally is in the range of 2-6 mm. The length of the impeller housing 36 can be equal to the length of the impeller head 22, approximately 1 mm for an axially fixed impeller head; or greater than the length of the impeller head 22. For example, for an axially fixed impeller head 22, the impeller housing 36 can have a length of approximately 1 mm; while for an axially moveable impeller head 22, the impeller housing can have a length of approximately 150 mm or 200 mm, a length equal to the length of a long lesion.

The diameter of the impeller housing 36 can be the same or different than the diameter of the housing shaft 64. In one embodiment, the diameter of the impeller housing 36 is greater than the diameter of the housing shaft 64 (see e.g. FIGS. 2 and 5). In one embodiment a transition region 66 is positioned between the impeller housing 36 and the housing shaft 64 and has a diameter that tapers from the diameter of the impeller housing 36 to the diameter of the housing shaft 64 (see e.g. FIGS. 2 and 5). The transition region 66 can be considered to form a part of either the impeller housing 36 or the housing shaft 64. The impeller housing 36 can have a tapered distal end region (e.g. FIGS. 5-6) or a non-tapered distal end region. In one aspect, the tapered distal end region includes a first tapered section and a second tapered section extending from the first tapered section to the distal end of the impeller housing 36 where the second tapered section extends at a different angle relative to the longitudinal axis of the drug delivery device than the first tapered section (e.g. FIGS. 5-6).

The number of the side openings 38 and/or the size of the side openings 38 of the impeller housing 36 control the rate of drug release and force of the drug. For example, a greater quantity of drug can exit through larger side openings and/or a greater number of side openings as compared to smaller side openings and/or a smaller number of side openings.

In one embodiment, the impeller housing 36 has only one side opening (not shown). The single side opening can have a circumferential extent of about 50% to 98% of the circumference of the impeller housing, and a longitudinal extent less than the longitudinal extent of the impeller housing 36 so that the wall of the impeller housing 36 bounds each side of the side opening.

In another embodiment, the impeller housing 36 has a plurality of side openings 38 (see e.g. FIGS. 2 and 4-6). The side openings 38 of the impeller housing 36 are regularly distributed about the circumference of the impeller housing 36. Each side opening 38 of the impeller housing 36 have a longitudinal extent less than the longitudinal length of the impeller housing 36, and a circumferential width. The side openings 38 can be continuous, or discontinuous (see e.g. FIG. 4, circumferential struts separating adjacent openings 38). A discontinuous side opening can also be described as a plurality of side openings. The continuous side openings can be oriented longitudinally or helically. The discontinuous side openings can be circumferentially aligned (same circumferential position, as shown e.g. FIG. 4) or circumferentially staggered (different circumferential positions, e.g. helically arranged, not shown). It is noted that even if drug bounces off the inner surface of the impeller housing, since the drug will be continually propelled by the impeller head and can eventually exit through the side openings. Even if not all of the drug exits through the side openings and thus is unused/undelivered drug, the rate of unused drug can be experimentally determined and factored in the dosage and/or amount of drug advanced to the impeller head.

In a further aspect, a drug delivery device 10 with an impeller 20 as described above includes a drug delivery mechanism 30 to advance the drug through the drug lumen 26 to the impeller head 22. Examples of suitable drug delivery mechanisms 30 include pressurized CO₂ gas; saline (agitated or non-agitated); a plunger 70 (e.g. FIG. 6); a syringe 72 (e.g. FIG. 3); a slurry; and combinations thereof. For example, a syringe can be used in combination with saline or a slurry. In one embodiment the pressurized CO₂ gas is provided in a canister. In yet a further aspect, the drug delivery mechanism 30 can include, or be in communication with, a drug reservoir. For example, syringe 72 includes a drug reservoir.

The drug delivery mechanism 30 can be detachable or non-detachable from the impeller 20. For example, the syringe 72 or the container with pressurized CO₂ gas can be detachable from the impeller 20, while the plunger 70 can be either detachable or non-detachable from the impeller 20. With regard to the syringe 72, when the syringe 72 is attached to the impeller 20 the syringe 72 is in fluid communication with the drug lumen 26 of the impeller 20 (e.g. FIG. 3).

With regard to the plunger 70, the plunger 70 is positioned in the drug lumen 26 and is independently slidable (e.g. plunger 70, shown in FIG. 6). For this embodiment, the drug 32 is inserted into the drug lumen at a location distal to the distal end of the plunger so that the plunger can advance the drug 32 through the drug lumen to the impeller head 22. For the plunger 70 shown in FIG. 6, the plunger 70 is independently slidable relative to the impeller shaft 24 and the housing shaft 64 for example by manipulation of a handle by the user of the drug delivery device. The plunger 70 has a head at the distal end that occludes the drug lumen in order to advance the entirety of the drug 32 in the drug lumen to the impeller head 22 (see e.g. FIG. 6). The head of the plunger 70 can have any suitable shape that occludes the drug lumen. In one aspect, an inner lumen defined by the plunger 70 is equal to the outer diameter of the impeller shaft 24 (e.g. FIG. 6). In a further aspect, only the head of the plunger 70 defines an inner lumen having a diameter equal to the outer diameter of the impeller head. In a further aspect, the surface of wall defining the inner lumen of the plunger 70 is lubricious to facilitate sliding. For example the wall can be formed of a lubricious material or have a lubricious layer or coating on the surface of the wall. Any suitable lubricious materials may be used, for example but not limited to Teflon; high density polyethylene (HDPE); silicone; hydrophilic coatings involving hydrogel polymers or the like, such as polymer networks of a vinyl polymer and an uncrosslinked hydrogel, for example; polyethylene oxide (PEO) (an example of a suitable hydrogel polymer); neopentyl glycol diacrylate (NPG) (an example of a vinyl polymer); and combinations thereof. U.S. Pat. No. 6,165,158, incorporated by reference in its entirety, discloses a shaft formed of a lubricious material and methods to form a shaft of lubricious material.

In a further aspect, a drug delivery device 10 with an impeller 20 as described above includes at least one expandable region 40 (schematically shown e.g. in FIGS. 4-6). In at least one embodiment, the at least one expandable region 40 temporarily blocks blood flow during delivery of the drug 32 by the drug delivery catheter 10 and/or dilates the lumen prior to drug delivery. In these embodiments, the loss of drug downstream is minimized.

The expandable region 40 can form a part of the impeller housing 36 (see e.g. FIGS. 5-6); a part of the housing shaft 64; a part of the exterior shaft 44 external to the housing shaft 64 (see e.g. in FIG. 4); or combinations thereof. The expandable region 40 can form only a portion of the shaft wall thickness (e.g. as schematically shown in FIGS. 4-6), or form an entirety of the shaft wall thickness.

In some embodiments, the drug delivery device 10 has only one expandable region 40. The single expandable region 40 can be positioned proximal to the impeller head 22 (see e.g. FIG. 4); or distal to the impeller head 22 (not shown). In other embodiments, the drug delivery device 10 has two expandable regions 40 with one expandable region positioned 40 proximal to the impeller head 22 and another expandable region 40 positioned distal to the impeller head 22 (see e.g. FIGS. 5-6).

The expandable region 40 can be an inflatable (e.g. a balloon), or actuatable (e.g. formed of electroactive polymer or a material with shape memory properties). With regard to the inflatable balloon, an inflation lumen is in fluid communication with the balloon for inflation of the balloon as is known in the art. Any suitable medical balloon material can be used for the inflatable balloon, for example but not limited to, nylon; polyamines; ethylene-vinyl acetate, polyvinyl chloride (PVC), olefin copolymers or homopolymers; polyethylenes; polyurethanes; crosslinked low density polyethylenes (PETs); highly irradiated linear low density polyethylene (LDPE); acrylonitrile polymers and copolymers; acrylonitrile blends; ionomer resins; polyethylene terephthalates; polyacrylenesulfide; and copolyesters. Electroactive polymers and catheters comprising electroactive polymers are discussed in U.S. Pat. No. 7,766,896; U.S. Pat. No. 7,909,844; U.S. Pat. No. 8,414,632; US 2005/0165439; and US 2007/0118169, each of which is incorporated by reference in its entirety. One example of a balloon formed of a material with shape memory properties is discussed in US 2004/0181252, incorporated by reference in its entirety.

In further aspect, the drug delivery device 10 as described above may include one or more areas, bands, coatings, members, etc. that is (are) detectable by imaging modalities such as X-Ray, MRI, ultrasound, etc. In some embodiments at least a portion of the drug delivery device 10 is at least partially radiopaque.

The drug 32 which can be delivered by a drug delivery device 10 as described herein can be any therapeutic agent or substance that has therapeutic benefit for local administration. Specific examples of drugs 32 include anti-restenosis drugs; anti-angiogenic drugs; paclitaxel; rapamycin; everolimus; and mixtures thereof.

In a further aspect, at least some of the drug 32 to be delivered by the drug delivery device 10 is in the form of drug crystals or crystalline forms of the drug 32. For example, the crystalline forms of paclitaxel include the anhydrous crystalline form and the crystalline dehydrate form. Any suitable method for forming drug crystals may be used. Exemplary methods of forming drug crystals are disclosed in US 2013/0035483; U.S. Pat. No. 7,820,812; and in EP 0717041, the entirety of each are incorporated by reference. Further, the drug crystals can be nanocrystals. Formation of nanocrystals is disclosed for example in US 2011/0008260, the entirety of which is incorporated by reference. As is known in the art, a crystalline drug formulation can include amorphous content. In other words, the drug can be a mixture of amorphous and crystalline forms of the drug. A drug 32 in the form of drug crystals is advanced by a drug delivery mechanism 30 that does not dissolve the drug crystal.

Exemplifications of a drug delivery device 10 as described above are provided in the following non-limiting examples.

### Example 1

FIG. 4 shows a first example of a drug delivery device 10 comprising an impeller 20 as described above. The drug delivery device 10 comprises an impeller 20 and an exterior shaft 44 with an expandable region 40, the exterior shaft 44 positioned around the housing shaft 64 of the impeller, and the expandable region 40 is positioned in the distal end region of the exterior shaft 44. In this example, the impeller housing 36 has a closed distal end. In one aspect the drug delivery device 10 is advanced to a treatment site through a sheath, guide catheter, introducer, endoscope, or other suitable device.

In one embodiment the drug lumen 26 is defined by the housing shaft 64 and impeller housing 36. When this embodiment is in use, the drug delivery mechanism 30 advances the drug 32 through the drug lumen 26 defined by the housing shaft 64 to the lumen defined by the impeller housing 36, where the centrifugal force of the rotating impeller head 22 positioned in the lumen of the impeller housing 36 drives the drug 32 out of the lumen of the impeller housing 36, through the side openings 38 of the impeller housing 36, and into the vessel wall 8. The distal end of the exterior shaft 44 is proximal to the impeller housing 36 so that the exterior shaft 44 does not cover the side openings 38 of the impeller housing 36 (see e.g. FIG. 4). During advancement of the drug delivery device, the exterior shaft 44 may cover the impeller housing 36 and be withdrawn prior to drug delivery. Thus, the exterior shaft 44 is longitudinally moveable relative to the housing shaft 64 and the impeller housing 36. If desired, the expandable region 40 can dilate the lumen prior to drug delivery.

In another embodiment, the drug delivery mechanism advances the drug 32 through the drug lumen 26 defined by the impeller shaft 24 and the impeller head 22. For this embodiment, the drug 32 exit the distal opening of the drug lumen 26 and into the lumen of the impeller housing 36 where the centrifugal force of the rotating impeller head 22 positioned in the lumen of the impeller housing 36 drives the drug 32 out of the lumen of the impeller housing 36, through the side openings 38 of the impeller housing 36, and into the vessel wall 8.

### Example 2

FIG. 5 shows a second example of a drug delivery device 10 comprising an impeller 20 as described above. The drug delivery device 10 comprises an impeller 20 with an impeller head 22 and an impeller shaft 24, the impeller shaft 24 and impeller head 22; a housing shaft 64 engaged to an impeller housing 36, the impeller housing 36 having two expandable regions 40a, 40b and side openings 38 positioned between the two expandable regions 40a, 40b; a drug lumen 26 defined by the housing shaft 64; a guidewire lumen 52 for a guidewire 50, the guidewire lumen 52 defined by the impeller shaft 24, the impeller head 22, and the impeller housing 36.

In use, the drug delivery mechanism advances the drug 32 through the drug lumen 26 defined by the housing shaft 64 into the lumen defined by the impeller housing 36 where the centrifugal force of the rotating impeller head 22 positioned in the lumen of the impeller housing 36 drives the drug 32 out of the lumen of the impeller housing 36, through the side openings 38 of the impeller housing 36, and into the vessel wall 8.

In a further aspect, the drug delivery device shown in FIG. 5 can include an exterior shaft with an expandable region as shown for the drug delivery device of FIG. 4 (not shown). In this embodiment, the expandable region can dilate the lumen before the drug is delivered.

### Example 3

FIG. 6 shows a third example of a drug delivery device 10 comprising an impeller 20 as described above. The drug delivery device 10 has the structures of the drug delivery device 10 of Example 2 (20, 22, 24, 26, 36, 40a, 40b, 50, 52, 64), except that the drug delivery mechanism is a plunger 70 positioned in the drug lumen 26 defined by the housing shaft 64.

In use, the plunger 70 advances the drug 32 through the drug lumen 26 defined by the housing shaft 64 into the lumen defined by the impeller housing 36 where the centrifugal force of the rotating impeller head 22 positioned in the lumen of the impeller housing 36 drives the drug 32 out of the lumen of the impeller housing 36, through the side openings 38 of the impeller housing 36, and into the vessel wall 8. In one embodiment the user moves a plunger handle (not shown) in a distal direction to advance the plunger 70 distally thereby advancing the drug 32 through the drug lumen 26 to the lumen defined by the impeller housing.

In a further aspect, the drug delivery device shown in FIG. 6 can include an exterior shaft with an expandable region as shown for the drug delivery device of FIG. 4 (not shown). In this embodiment, the expandable region can dilate the lumen before the drug is delivered.

### Method of Use

A drug delivery device 10 as described herein can be used to deliver a drug to a target site. Aspects of the drug to be delivered are discussed above in greater detail. In one aspect, a method of delivering the drug comprises: advancing a drug delivery device as described herein to a target site; advancing drug to the impeller head of the drug delivery device; and rotating the impeller head to deliver the drug into the target site. In another aspect, a method of delivering a drug to a body lumen comprises: advancing a drug delivery device as described herein to a target site in a body lumen; advancing a drug through the drug lumen to the impeller head of the drug delivery device; and delivering the drug into the target site by centrifugal force. In yet another aspect, a method of delivering a drug to a body lumen comprises: advancing a drug delivery device as described herein to a target site in a body lumen; advancing a drug through the drug lumen to the impeller head of the drug delivery device; and applying a centrifugal force to the drug wherein the centrifugal force delivers the drug into the target site. In a further aspect, a method of delivering a drug to a body lumen comprises: advancing a drug delivery device as described herein to a target site in a body lumen; advancing a drug through the drug lumen to the impeller head of the drug delivery device; and driving the drug radially outward to the target site.

It is noted that: the impeller head can begin rotation before the drug reaches the impeller head; an expandable region of the drug delivery device can dilate the lumen before delivering the drug; and/or an expandable region of the drug delivery device can block blood flow as the drug is delivered into the target site.

The embodiments or aspects of the drug delivery device discussed above and presented in the claims, may be combined in any fashion and combination as shown for example by the nonlimiting embodiments or aspects presented in the following statements:

Statement 1. A drug delivery device comprising:

an impeller, the impeller comprising:
an impeller housing positioned at a distal end of a housing shaft, the impeller housing and the housing shaft each defining a lumen, the impeller housing comprising a side opening for passage of a drug therethrough;
a rotatable impeller head positioned at a distal end of an impeller shaft, the impeller head positioned in the lumen of the impeller housing, the impeller shaft positioned in the lumen defined by the housing shaft;
a drug lumen for passage of a drug to the rotatable impeller head for delivery into a lumen wall by the rotatable impeller.

Statement 2. The drug delivery device of Statement 1, wherein the impeller housing has a closed distal end and the drug lumen is defined by the housing shaft and the impeller housing.

Statement 3. The drug delivery device of Statement 2, the impeller further comprising:

an exterior shaft positioned around the housing shaft, the exterior shaft including an expandable region, wherein the exterior shaft is longitudinally moveable relative to the impeller housing.

Statement 4. The drug delivery device of Statement 1, wherein the impeller housing has two expandable regions, the side opening is positioned between the two expandable regions, and the drug lumen is defined by the housing shaft.

Statement 5. The drug delivery device of Statement 4, wherein a guidewire lumen is defined by the impeller shaft, the impeller head, and the impeller housing.

Statement 6. The drug delivery device of any one of Statements 4-5, the drug delivery device further comprising an exterior shaft positioned around the housing shaft, the exterior shaft including an expandable region, wherein the exterior shaft is longitudinally moveable relative to the impeller housing.

Statement 7. The drug delivery device of any one of Statements 4-6, the impeller further comprising a plunger positioned in the drug lumen.

Statement 8. The drug delivery device of Statement 7, the plunger comprising a plunger handle for independent movement of the plunger.

Statement 9. The drug delivery device of any one of Statements 1-8, wherein the impeller shaft is rotatable.

Statement 10. The drug delivery device of any one of Statements 1-8, wherein the impeller shaft is not rotatable.

Statement 11. The drug delivery device of any one of Statements 1-10, wherein the side opening is a plurality of side openings.

Statement 12. The drug delivery device of any one of Statements 1-10, wherein the side opening is a single side opening.

Statement 13. The drug delivery device of Statement 12, wherein the single side opening has a circumference extent of about 50-98% of a circumference of the impeller housing.

Statement 14. The drug delivery device of any one of Statements 1-13, wherein the impeller head has a smooth outer surface.

Statement 15. The drug delivery device of any one of Statements 1-13, wherein the impeller head has a textured outer surface.

Statement 16. The drug delivery device of Statement 15, the textured outer surface includes at least one protuberance.

Statement 17. The drug delivery device of Statement 16, the at least one protuberance in the form of ribs, cleats, posts, bumps, and combinations thereof.

Statement 18. The drug delivery device of Statement 17, the protuberance being a rib.

Statement 19. The drug delivery device of Statement 18, wherein the rib is a plurality of ribs.

Statement 20. The drug delivery device of any one of Statements 17-19, where the rib is straight.

Statement 21. The drug delivery device of any one of Statements 17-20, wherein the rib is oriented longitudinally.

Statement 22. The drug delivery device of any one of Statements 17-20, wherein the rib is curved.

Statement 23. The drug delivery device of any one of Statements 17-22, wherein the rib is oriented helically.

Statement 24. The drug delivery device of any one of Statements 15-23, wherein a distal face of the impeller head includes the textured outer surface.

Statement 25. The drug delivery device of any one of Statements 17-24, wherein a number of the side opening of the impeller housing is equal to a number of the rib.

Statement 26. The drug delivery device of any one of Statements 1, and 9-25, wherein the impeller further comprises a guidewire lumen for passage of a guidewire.

Statement 27. The drug delivery device of Statement 26, wherein the guidewire lumen is defined in part by the impeller shaft.

Statement 28. The drug delivery device of any one of Statements 1 and 9-27, wherein the drug lumen is defined in part by the impeller shaft.

Statement 29. The drug delivery device of any one of Statements 1 and 9-28, wherein the drug lumen is defined in part by the housing shaft.

Statement 30. The drug delivery device of any one of Statements 1 and 9-29, further comprising a drug delivery mechanism to advance the drug to the impeller head.

Statement 31. The drug delivery device of Statement 30, wherein the drug delivery mechanism is selected from the group consisting of pressurized CO₂ gas; saline; agitated saline; a plunger; a syringe; a slurry; and combinations thereof.

Statement 32. The drug delivery device of Statement 31, wherein the drug delivery mechanism is non-detachable.

Statement 33. The drug delivery device of any one of Statements 31-32, wherein the drug delivery mechanism is a plunger positioned in the drug lumen.

Statement 34. The drug delivery device of any one of Statements 31-33, wherein the drug lumen is defined in part by the housing shaft, the plunger being independently slidable relative to the housing shaft and to the impeller shaft for advancing a drug through the drug lumen to the impeller head.

Statement 35. The drug delivery device of any one of Statements 31-34, wherein the plunger includes a handle at a proximal end.

Statement 36. The drug delivery device of Statement 31, wherein the drug delivery mechanism is detachable.

Statement 37. The drug delivery device of any one of Statements 31 and 36, wherein the drug delivery mechanism is a syringe in fluid communication with the drug lumen.

Statement 38. The drug delivery device of any one of Statements 31 and 36, wherein the drug delivery mechanism is pressurized CO₂ gas contained in a canister.

Statement 39. The drug delivery device of any one of Statements 1 and 9-38, further comprising a first expandable region positioned proximal to the rotatable impeller head.

Statement 40. The drug delivery device of Statement 39, further comprising a second expandable region positioned distal to the rotatable impeller head.

Statement 41. The drug delivery device of any one of Statements 39-40, wherein the expandable region has a thickness equal to or less than a wall thickness.

Statement 42. The drug delivery device of any one of Statements 39-41, wherein the first and second expandable regions form a part of the impeller housing.

Statement 43. The drug delivery device of any one of Statements 39 and 41, further comprising an exterior shaft defining a lumen, the housing shaft positioned in the lumen of the exterior shaft, wherein the first expandable region forms a part of the exterior shaft.

Statement 44. The drug delivery device of any one of Statements 39 and 41, further comprising an exterior shaft defining a lumen, the housing shaft positioned in the lumen of the exterior shaft, the exterior shaft comprising the first expandable region.

Statement 45. The drug delivery device of any one of Statements 39-44, wherein the expandable region(s) is/are selected from the group consisting of inflatable balloons and electroactive polymers.

Statement 46. The drug delivery device of any one of Statements 39 and 41-45, wherein the first expandable region is an inflatable balloon.

Statement 47. The drug delivery device of any one of Statements 39 and 41-45, wherein the first expandable region comprises an actuatable material.

Statement 48. The drug delivery device of Statement 47, wherein the actuatable material is selected from the group consisting of electroactive polymers, materials with shape memory properties, and combinations thereof.

Statement 49. The drug delivery device of any one of Statements 39 and 41-45, wherein the first expandable region comprises a material with shape memory properties.

Statement 50. The drug delivery device of any one of Statements 40-44, wherein the first and second expandable regions of the impeller housing and the expandable region of the exterior shaft are inflatable balloons, actuatable materials, and combinations thereof.

Statement 51. The drug delivery device of Statement 50, wherein the first and second expandable regions of the impeller housing and the expandable region of the exterior shaft are actuatable materials, the actuatable materials selected from the group consisting of electroactive polymers, materials with shape memory properties, and combinations thereof.

Statement 52. The drug delivery device of Statement 50, wherein the first and second expandable regions of the impeller housing and the expandable region of the exterior shaft are inflatable balloons.

Statement 53. The drug delivery device of any one of Statements 1-52, further comprising a mechanism to rotate the impeller.

Statement 54. The drug delivery device of Statement 53, wherein the mechanism to rotate the impeller is detachably connected to the impeller.

Statement 55. The drug delivery device of Statement 53, wherein the mechanism to rotate the impeller forms a part of the impeller and is not detachably connected to the impeller.

Statement 56. The drug delivery device of any one of Statements 1 and 4-55, wherein the impeller is over-the-wire and includes a guidewire lumen extending from a proximal end of the impeller to a distal end of the impeller.

Statement 57. The drug delivery device of any one of Statements 1 and 4-55, wherein the impeller is monorail and includes a guide wire lumen extending for only a portion of a longitudinal length of the impeller.

Statement 58. The drug delivery device of any one of Statements 1-57, wherein the impeller head has a longitudinal length of approximately 1 to 10 mm.

Statement 59. The drug delivery device of Statement 58, wherein the longitudinal length of the impeller head is 1 mm.

Statement 60. The drug delivery device of any one of Statements 1-59, wherein the impeller shaft and impeller head are longitudinally moveable relative to the housing shaft and impeller housing.

Statement 61. The drug delivery device of any one of Statements 1-59, wherein the impeller shaft and impeller head are longitudinally fixed relative to the housing shaft and the impeller housing.

Statement 62. The drug delivery device of any one of Statements 1-61, the impeller housing having a longitudinal length of approximately 150 to 200 mm.

Statement 63. The drug delivery device of any one of Statements 1-59 and 61, the impeller housing having a longitudinal length of approximately 1 mm.

Statement 64. A method of delivering a drug to a body lumen comprising:

advancing the drug delivery device of any one of Statements 1-63 to a target site in a body lumen;

advancing a drug through the drug lumen to the impeller head of the drug delivery device; and

rotating the impeller head to deliver the drug into the target site.

Statement 65. A method of delivering a drug to a body lumen comprising:

advancing the drug delivery device of any one of Statements 1-63 to a target site in a body lumen;

advancing a drug through the drug lumen to the impeller head of the drug delivery device; and

delivering the drug into the target site by centrifugal force.

Statement 66. A method of delivering a drug to a body lumen comprising:

advancing the drug delivery device of any one of Statements 1-63 to a target site in a body lumen;

advancing a drug through the drug lumen to the impeller head of the drug delivery device; and

applying a centrifugal force to the drug, wherein the centrifugal force delivers the drug into the target site.

Statement 67. The method of any one of Statements 64-66, wherein the drug comprises a crystalline form of the drug.

Statement 68. The method of any one of Statements 64-67, wherein impeller has a smooth outer surface and the medium delivering the drug is viscous.

Statement 69. The method of Statement 68, the medium having a viscosity of 0.01 to 50,000 cP.

Statement 70. The method of any one of Statements 64 and 67-69, wherein the advancing and rotating steps are executed concurrently.

Statement 71. The method of any one of Statements 64-69, wherein the step of rotating the impeller head begins before the drug reaches the impeller head.

Statement 72. The method of any one of Statements 64-71, wherein the drug delivery mechanism of any one of Statements 31-38 is used for advancing the drug through the drug lumen to the impeller head.

Statement 73. The method of any one of Statements 64-72, further comprising dilating the lumen with the drug delivery device before advancing the drug through the drug lumen to the impeller head.

Statement 64. The method of Statement 73, wherein an expandable region of any one of Statements 39-52 is used for dilating the lumen.

Statement 75. The method of any one of Statements 64-74, wherein a motor drive rotates the impeller head.

Statement 76. The method of any one of Statements 64-75, further comprising advancing or retracting the impeller head along the target site.

Statement 77. The method of any one of Statements 64-76, wherein the drug delivery device is advanced to the target site through a secondary medical device, the secondary medical device selected from the group consisting of guide catheter, sheath, and endoscope.

Statement 78. The method of any one of Statements 64-76, wherein the drug delivery device is advanced to the target site over a guide wire.

Statement 79. The method of any one of Statements 64-78, further comprising blocking blood flow as the drug is delivered into the target site.

Statement 80. The method of Statement 79, wherein an expandable region of any one of Statements 39-51 is used to block blood flow.

Statement 81. The method of any one of Statements 66-80, wherein a rotating impeller head applies the centrifugal force to the drug.

Statement 82. The method of any one of Statements 64-81, wherein the drug is a mixture of amorphous and crystalline forms of the drug.

Statement 83. The method of Statement 82, wherein at least 50% of the drug is in a crystalline form.

Statement 84. The method of any one of Statements 82-83, wherein at least 60% of the drug is in a crystalline form.

Statement 85. The method of any one of Statements 82-84, wherein at least 70% of the drug is in a crystalline form.

Statement 86. The method of any one of Statements 82-85, wherein at least 80% of the drug is in a crystalline form.

Statement 87. The method of any one of Statements 82-86, wherein at least 90% of the drug is in a crystalline form.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1. A drug delivery device comprising:
an impeller, the impeller comprising:
an impeller housing positioned at a distal end of a housing shaft, the impeller housing and the housing shaft each defining a lumen, the impeller housing comprising a side opening for passage of a drug therethrough;
a rotatable impeller head positioned at a distal end of an impeller shaft, the impeller head positioned in the lumen of the impeller housing, the impeller shaft positioned in the lumen defined by the housing shaft;
a drug lumen for passage of a drug to the rotatable impeller head for delivery into a lumen wall by the rotatable impeller.

2. The drug delivery device of claim 1, wherein the impeller shaft is rotatable.

3. The drug delivery device of any one of claim 1, wherein the side opening is a plurality of side openings.

4. The drug delivery device of claim 1, wherein the impeller head has a rib for directing a drug to a lumen wall.

5. The drug delivery device of claim 4, wherein the rib is a plurality of ribs.

6. The drug delivery device of claim 1, wherein the impeller further comprises a guidewire lumen for passage of a guidewire.

7. The drug delivery device of claim 6, wherein the guidewire lumen is defined in part by the impeller shaft.

8. The drug delivery device of claim 1, wherein the drug lumen is defined in part by the impeller shaft.

9. The drug delivery device of claim 1, wherein the drug lumen is defined in part by the housing shaft.

10. The drug delivery device of claim 1, further comprising a drug delivery mechanism to advance the drug to the impeller head.

11. The drug delivery device of claim 10, wherein the drug delivery mechanism is selected from the group consisting of pressurized CO₂ gas; saline; agitated saline; a plunger; a syringe; a slurry; and combinations thereof.

12. The drug delivery device of claim 11, wherein the drug delivery mechanism is a plunger, the plunger being positioned in the drug lumen.

13. The drug delivery device of claim 11, wherein the drug delivery mechanism is a syringe, the syringe being in fluid communication with the drug lumen.

14. The drug delivery device of claim 1, the impeller further comprising a first expandable region positioned proximal to the rotatable impeller head.

15. The drug delivery device of claim 14, the impeller further comprising a second expandable region positioned distal to the rotatable impeller head.

16. The drug delivery device of claim 15, wherein the first and second expandable regions form a part of the impeller housing.

17. The drug delivery device of claim 14, further comprising an exterior shaft defining a lumen, the housing shaft positioned in the lumen of the exterior shaft, wherein the first expandable region forms a part of the exterior shaft.

18. The drug delivery device of claim 14, wherein the first expandable region is selected from the group consisting of inflatable balloons and electroactive polymers.

19. A method to deliver a drug to a lumen wall comprising
advancing a drug delivery device to a treatment site, the drug delivery device comprising:
an impeller, the impeller comprising:
an impeller housing positioned at a distal end of a housing shaft, the impeller housing and the housing shaft each defining a lumen, the impeller housing comprising a side opening for passage of a drug therethrough;
a rotatable impeller head positioned at a distal end of an impeller shaft, the impeller head positioned in the lumen of the impeller housing, the impeller shaft positioned in the lumen defined by the housing shaft;
a drug lumen for passage of a drug to the rotatable impeller head;
advancing a drug through the drug lumen to the impeller head; and
rotating the impeller head to deliver the drug by centrifugal force.

20. The method of claim 19, wherein the drug comprises a crystalline form of the drug.
